(19) United States
(12) Patent Application Publication
(10) Pub. No.: US 2026/0166542 A1
(43) Pub. Date: Jun. 18, 2026

(54) COMPOSITIONS, METHODS, AND SYSTEMS FOR SAMPLE PROCESSING WITH MORPHOLOGY-ADJUSTABLE FUNCTIONALIZED PARTICLES

(71) Applicant: Bio-Rad Laboratories, Inc., Hercules, CA (US)

(72) Inventors: Vishal Sharma, Hercules, CA (US); Ronald Lebofsky, Hercules, CA (US); Man Cheng, Hercules, CA (US); Adam McCoy, Hercules, CA (US); Kalyan Handique, Hercules, CA (US); Eric Pomaranski, Hercules, CA (US)

(21) Appl. No.: 19/354,602

(22) Filed: Oct. 9, 2025

Related U.S. Application Data
(63) Continuation of application No. 18/076,667, filed on Dec. 7, 2022, now Pat. No. 12,465,910.
(60) Provisional application No. 63/288,405, filed on Dec. 10, 2021.

(51) Int. Cl. B01L 3/00; C12N 13/00

(52) U.S. Cl. CPC B01L 3/502; C12N 13/00; B01L 2200/026; B01L 2200/0668; B01L 2200/10; B01L 2200/16; B01L 2300/047; B01L 2300/0893

(57) ABSTRACT

Inventions covered include methods, systems, and compositions for sample processing, involving morphology-adjustable (e.g., tunable on-demand) functionalized particles. In some embodiments, a method can include distributing a set of functionalized particles, in a first morphological state, across a set of partitions; transitioning the set of functionalized particles, at the set of partitions, from the first morphological state to a second morphological state; transitioning the set of functionalized particles, at the set of partitions, from the second morphological state to a third morphological state, and inducing interactions between the set of functionalized particles and a set of targets, within the set of partitions and according to a set of operations with a set of process fluids.

### CROSS-REFERENCE TO RELATED APPLICATIONS

This application claims the benefit of U.S. Provisional Application No. 63/288,405 filed on 10 Dec. 2021, which is incorporated in its entirety herein by this reference.

### TECHNICAL FIELD

This invention relates generally to the target capture and processing field, and more specifically to new and useful compositions, methods, and systems for sample processing using morphology-adjustable functionalized particles.

### BACKGROUND

With an increased interest in biological target capture and characterization, partitioning platforms that allow for isolation, identification, and/or retrieval of targets are becoming highly desirable. Such platforms can support applications of use involving target amplification (e.g., digital PCR) and characterization of spatial distributions of targets (e.g., with respect to spatial transcriptomics), by implementing functionalized compounds that are structured to capture target material for subsequent detection. However, such platforms often require complex workflows with manual steps prone to user error. Furthermore, such platforms provide limited control of reactions related to target capture, target release, and target retrieval. Additionally, compositions of materials can be improved significantly for applications involving capture and retrieval of target material in a manner that allows for analysis of single cells, material derived from single cells, and/or other targets from biological samples. As such, these processes require extensive and iterative manual library preparation and selection processes, may not amenable to automation, and may thus result high background noise rates, elevated false positive rates, or otherwise unreliable experimental results.

Thus, there is a need in the target capture and processing field to create new approaches to target capture, release, and/or detection. The invention(s) described herein cover new and useful compositions, methods, and systems for sample processing using morphology-adjustable functionalized particles.

### BRIEF DESCRIPTION OF THE FIGURES

FIG. 1A depicts a schematic of an embodiment of a method for sample processing with morphology-adjustable functionalized particles.

FIG. 1B depicts a schematic of a variation of a method for sample processing with morphology-adjustable functionalized particles.

FIGS. 2A and 2B depict examples of steps for preparing and distributing particles across partitions.

FIG. 3 depicts examples of steps for reconstituting particles to produce one or more morphological states suitable for sample processing and retrieval.

FIG. 4 depicts examples of steps for producing one or more morphological states suitable for particle swelling and retrieval.

FIGS. 5A-5C depict variations of operational modes attributed to different controllable states of morphology-adjustable functionalized particles.

FIG. 6 depicts example results of assays performed using morphology-adjustable functionalized particles.

FIG. 7 depicts example results of sequencing processes performed using morphology-adjustable functionalized particles.

FIG. 8 depicts a schematic of an embodiment of a morphology-adjustable functionalized particle.

FIGS. 9A and 9B depict variations of using morphology-adjustable functionalized particles as semi-permeable membranes.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Benefits

The invention(s) described can confer several benefits over conventional systems, methods, and compositions.

In particular, the inventions provide morphology-tunable functionalized particles that can be used for pre-loading of microwell substrates and/or other substrates, thereby providing a higher degree of efficiency in relation to distribution (e.g., in a manner that requires fewer particles overall, such as 50% or less than current methods for particle loading of partitions, to achieve desired distributions of particles across partitions), and contributing to less material waste. In a specific example, the methods described can distribute particles across wells (e.g., microwells) of a substrate with use of ˜50% fewer particles in comparison to methods of distribution where particles are loaded within wells in contemporaneously with sample distribution to the set of wells, to achieve an approximately 1:1 distribution of particles to wells. Alternatively, the invention(s) described can achieve another desired controlled distribution of particles to wells (e.g., less than 1 particle per well on average, 2 particles per well, 3 particles per well, etc.).

Alternatively, the invention(s) can be used to pre-load microwell substrates with particles in a desired pattern. For instance, the microwell substrate can be pre-loaded with a first particle type (e.g., configured to process a first type of target of a sample) in a first pattern or at a first subregion of the microwell substrate, and with a second particle type (e.g., configured to process a second type of target of the sample) in a second pattern or at a first subregion of the microwell substrate, such that distinct target types of the sample can be differentially captured, processed, and/or detected based upon the predetermined first pattern, second pattern, first subregion, or second subregion of the microwell substrate.

Relatedly, the inventions also provide improved methods for enabling co-capture of functionalized particles with target material (e.g., single cells, nucleic acids, nucleic acid fragments, proteins, small molecules, other analytes, etc.) in a one-to-one manner. As such, where the set of targets include nucleic acids of a set of single cells of the sample and where the set of partitions includes a set of microwells, the methods described can involve co-capturing the set of single cells with the set of functionalized particles within the set of partitions (e.g., with the set of functionalized particles in a second morphological state).

Still relatedly, the inventions provide improved methods for distributing/pre-loading functionalized particles across a distribution of partitions (e.g., microwell plate, other substrate, etc.), in a manner that results in higher efficiency of one particle per partition.

The inventions further enable applications related to adjusting morphology (e.g., sizes, shapes) of functionalized particles for target capture during loading onto a substrate, storage (e.g., with drying of particles), transportation, sample processing, and release from the substrate, in a highly controlled and on-demand manner. In some embodiments the desired organizational structure is achieved in a self-assembling manner due to the morphology. In relation to storage, the inventions can improve processes in a manner that provides better shelf life of components prior to use. The inventions can also reduce manufacturing burden in relation to usage and storage of materials and improve target capture workflows. In relation to drying and rehydrating of particles, the inventions further provide non-intuitive results in relation to initial and final morphologies of particles, thus enabling new highly-controllable applications of use.

In one use case, particles are loaded in a dehydrated state and then hydrated with desired osmolarity parameters in coordination with use of the substrate for sample processing, where hydrated particles are larger in diameter than their respective microwells. By tuning the size according to level of hydration and/or osmolarity, the particles may shrink to the point where they enter the microwell. The shrunken particle size may be such that only one bead can fit per microwell. Furthermore, upon hydration with desired osmolarity parameters, the particles may swell to seal off their respective microwells, and/or swell to push contents out of microwells for washing (e.g., in the context of waste material), and/or downstream retrieval and processing (e.g., in scenarios were targets of a sample are loaded above particles within microwells).

In another use case, the height of the microwell is identical/equal to the diameter (or other characteristic dimension) of the loaded particle (e.g., in a first morphological state). Further shrinking the loaded particle based upon environmental parameters (e.g., hydration, osmolarity, electric field, pH, temperature, etc.) can provide a volume for the loading of a substrate, i.e. a cell.

In another use case, the particle is designed to dissolve (e.g., transition to a fourth morphological state) upon the addition of an exogenous agent. For example the particle can comprise N, N′-Bis(acryloyl)cystamine, which dissolves when reacted with reducing agents such as DTT or TCEP.

In an exemplary use case where a dissolvable hydrogel is used for particle composition, the particle can be dried after loading. When loading a substrate such as a cell, the dissolving agent can be added such that upon rehydration the particle dissolves and thereby occupies a reduced or otherwise negligible amount of an internal volume of the microwell, thus allowing for the occupancy of the microwell by the substrate (e.g., a cell, another target).

The invention enables quality control in relation to the nature of loading particles within microwells of a substrate, since shrinking and/or drying the particles will reduce the physical overlap of one particle with another, thereby allowing single unit particle detection through imaging. Distinct boundaries between particles, for instance, can improve computational performance when imaging and/or otherwise detecting signals from contents of microwells.

In particular, the inventions can enable efficient methods for releasing particles from microwells and/or other substrate on-demand and in a rapid manner. Release and retrieval are typically difficult and non-efficient when using particles in high-aspect wells, due to close packing of wells of capture platforms. The ability to control workflow steps in which particles are retained within in partitions and workflow steps in which the particles (and attached content) are either removed or transitioned to another morphological state enables drastically improved workflows. Retrieval mechanisms described in relation to morphology-adjustable particles also subject target material to acceptable amounts of shear and other potential stresses that would otherwise obstruct downstream processing steps.

In specific applications, the inventions can be applied to partitions for digital PCR, other amplification assays, and/or other assays involving target capture and synthesis.

In specific applications, the inventions can be used to characterize spatial distributions of targets of a biological sample. For instance, controlled states of swelling of morphology-adjustable particles from addressable microwells can be used in the context of spatial transcriptomics, where, decoding positions of functionalized particles and captured targets (e.g., through sequencing) can be used to create spatial maps of captured targets. In one exemplary use case, particles can be loaded within microwells in a shrunken state, transitioned to a state in which they protrude from their respective microwells (or are otherwise able to contact sample material) by hydration with desired osmolarity factors, and interacting the substrate with a sample (e.g., tissue sample) with the particles can be used to determine relative spatial positions of targets of the tissue sample (e.g., single cells, single cell subtypes, proteins, nucleic acids such as RNAs and DNAs, other biomarkers).

The invention(s) confer(s) the benefit of providing non-naturally occurring compositions for facilitating capture, extraction, and/or retrieval of target biological material from a sample, while providing barcoding for each biomarker molecule retrieved from a partition of a sample. Barcoding and/or known addresses enable detection of particle positions, and thus, captured target positions, upon retrieving and sequencing processed genetic complexes generated using the particles. Furthermore, the invention(s) relate to combinations of materials, where the combinations of materials are non-naturally occurring (e.g., there is no naturally occurring counterpart to the compositions described and claimed).

The invention(s) also confer(s) the benefit of increasing the efficiency at which target material is retrieved (and non-target material is not retrieved). Selective retrieval efficiency can thus reduce downstream costs in relation to processing reagent and other material costs (due to reduced volumes needed), processing burden, and improved signal to noise ratios. In one example use case, osmotic pressure-induced swelling is proportional to charge of the particle; thus, particles without captured target material may swell less and thus remain in partitions, while particles with captured target material may swell more and thus become enriched in the population of particles released from partitions during downstream processing steps. As such, methods described can include: distributing a set of functionalized particles, in a first morphological state, across a set of partitions; co-capturing a set of targets of a sample with a first subset of functionalized particles; transitioning the first subset of the set of functionalized particles to a second morphological state (e.g., from the first morphological state, from a state intermediate to the first morphological state and the second morphological state, etc.) and a second subset of the set of functionalized particles (that have not captured targets of the sample) to a third morphological state (e.g., from the first morphological state, from a state intermediate to the first morphological state and the third morphological state, etc.) using a medium, where particles in the second morphological state have a higher degree of swelling in comparison to particles in the third morphological state; and preferentially releasing particles of the first subset from the set of partitions.

Alternatively, in a scenario where it is preferred to release particles that have no associated captured targets, methods described can include: distributing a set of functionalized particles, in a first morphological state, across a set of partitions; co-capturing a set of targets of a sample with a first subset of functionalized particles; transitioning the first subset of the set of functionalized particles to a second morphological state (e.g., from the first morphological state, from a state intermediate to the first morphological state and the second morphological state, etc.) and a second subset of the set of functionalized particles (that have not captured targets of the sample) to a third morphological state (e.g., from the first morphological state, from a state intermediate to the first morphological state and the third morphological state, etc.) using a medium, where particles in the second morphological state have a lower degree of swelling in comparison to particles in the third morphological state and remain within the set of partitions; and preferentially releasing particles of the second subset from the set of partitions.

The invention(s) also confer the benefit of enabling at least partial automation of the protocols involved in target capture (e.g., single cell capture), sample waste removal, target material retrieval, and subsequent processing. For instance, a human operator user can be removed from part or all of the method. For example, tunable particles can be loaded and dried during consumable manufacturing, removing this process from the user workflow, and facilitating the automation of downstream steps. Furthermore, the system(s) and/or method(s) can enable better accuracy in performance of a protocol over conventional systems and methods. Some of these inventions are also much more amenable to full automation with a liquid handling robot.

Additionally or alternatively, the invention(s) can confer any other suitable benefit.

### 2. Methods for Adjusting Morphology of Functionalized Particles and Applications of Use

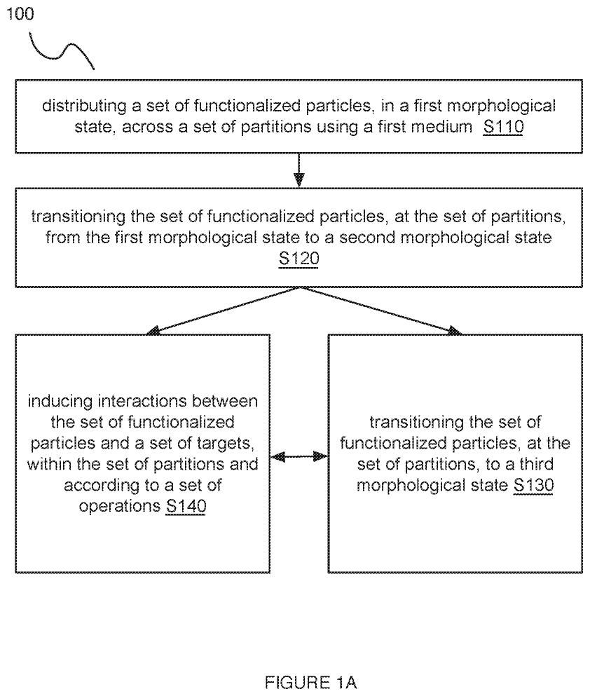

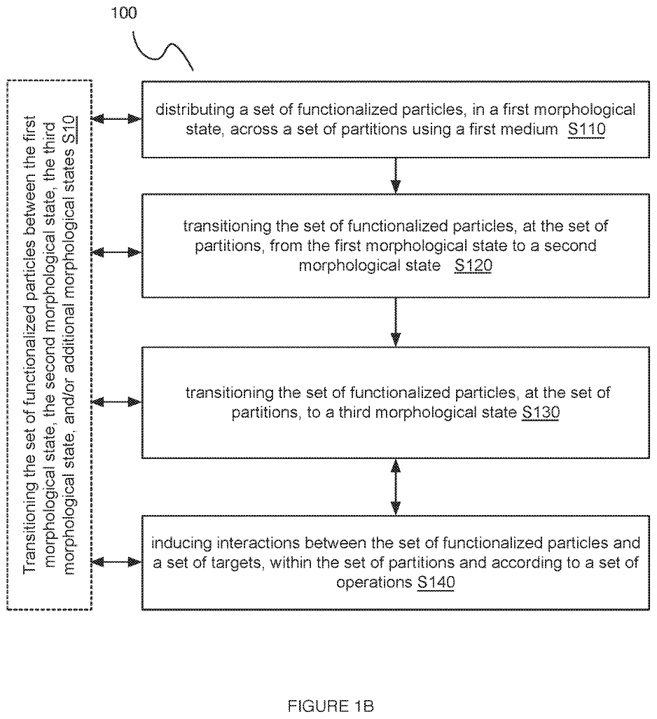

As shown in FIG. 1A, an embodiment of a method 100 includes: distributing a set of functionalized particles, in a first morphological state, across a set of partitions using a first medium S110; transitioning the set of functionalized particles, at the set of partitions, from the first morphological state to a second morphological state S120; and processing the set of partitions with one or more of a set of process fluids, thereby transitioning the set of functionalized particles, at the set of partitions, from the second morphological state to a third morphological state (e.g., using a second medium) S130.

In variations, the method 100 can further include inducing interactions between the set of functionalized particles and a set of targets, within the set of partitions and according to a set of operations with a set of process fluids S140, where step S140 can be performed prior to step S130, after step S130, or otherwise contemporaneously with (e.g., simultaneously with delivery of process fluids, near in time to delivery of process fluids, etc.) step S130.

In still other variations, as shown in FIG. 1B, the method 100 can further include transitioning the set of functionalized particles between the first morphological state, the second morphological state, the third morphological state, and/or additional morphological states S10, where, different states of functionalized particles are described in more detail below. As such, the method 100 can include coverage of transitions between different states (e.g., less than three states, more than three states, etc.) to provide functionality during various assays and/or steps of processing a sample.

The method 100 functions to implement and manipulate morphology-tunable functionalized particles, on-demand, in order to enable novel and non-obvious means for processing and characterizing targets from a sample. The method 100 can further be used for pre-loading of microwell substrates and/or other substrates in a manner that uses fewer functionalized particles, thereby providing a higher degree of efficiency in relation to distribution, and contributing to less material waste. In a specific example, the methods described can distribute particles across wells (e.g., microwells) of a substrate with use of ˜50% fewer particles in comparison to methods of distribution where particles are loaded within wells in contemporaneously with sample distribution to the set of wells, to achieve an approximately 1:1 distribution of particles to wells. Alternatively, the methods described can achieve another desired controlled distribution of particles to wells (e.g., less than 1 particle per well on average, 2 particles per well, 3 particles per well, etc.). The method 100 also enables co-capture of functionalized particles with target material (e.g., single cells), with improved processes that also provide better shelf life of components prior to use. The method 100 further provides non-intuitive results in relation to initial and final tuned morphologies of particles, thus enabling new highly-controllable applications of use.

In one use case, on-demand tuning of particle morphology/size can provide adjustable volumes (e.g., larger volumes, smaller volumes) for reactions to occur within a microwell containing a target (e.g., cell, cell-derived material, nucleic acid, protein, analyte, etc.) and a particle. Additionally, in a non-intuitive and non-obvious manner, initial distribution of particles (having a first morphological size) across a set of partitions using a medium, drying the distribution of particles within the partitions, and later reconstitution of the distribution of particles within the partitions in the same medium can produce particles larger than the first morphological size, which can facilitate release of particles from partitions in an efficient manner (e.g., where swelling causes particles to push out of microwells), along with other applications. Release and retrieval are typically difficult and non-efficient when using particles in high-aspect wells, due to close packing of wells of capture platforms; thus, the methods provide utility in a non-intuitive manner. Furthermore, addition of other process reagents during reconstitution can be used to control equilibrium particle morphology within a partition, with corresponding applications of use.

The methods described can be performed in arrays of wells (e.g., microwells, nanowells, nanovials, etc.), fluidic systems (e.g., including channels, chambers, other fluidic structures, etc.), tubes, or other suitable fluid containers. Additionally or alternatively, the methods described can be performed using substrates (e.g., slides, plates, other substrates) upon which functionalized particles can be applied (e.g., in droplet or spotted format). Additionally or alternatively, the methods described can be performed within other droplets or partitions (e.g., in the context of digital PCR or other partitioning) applications.

In one non-limiting example, the particles can be arrayed on a planar surface (e.g., a slide) in an enlarged state, where in the enlarged state, the particles self-arrange in a close packed array (e.g., hexagonal close packed array, rectangular close packed array). Subsequent attachment of the particles to the surface and decreasing the particle size results in effectively partitioning each of the particles (e.g., by creating an empty space between adjacent particles). Particles can subsequently be further separated by using air, oil, or other materials to fill the spaces between particles that arose due to the size change. As such, distributing the set of functionalized particles, in a first morphological state, can include distributing the set of functionalized particles in an enlarged state, and the method can further include: transitioning the set of functionalized particles from the enlarged state to a reduced state upon shrinking each of the set of functionalized particles (e.g., by dehydration, by an osmotic shift, etc.); and partitioning each of the set of functionalized particles from each other (e.g., based upon such shrinkage and/or with a filler medium positioned within empty spaces between adjacent particles of the set of functionalized particles.

The methods described can be implemented using embodiments, variations, and examples of system and microfluidic substrate components described in one or more of: U.S. Publication number 2018/0364148 filed 27 Jul. 2018; U.S. Publication number 2019/0144931 filed 30 Jul. 2018; U.S. Pat. No. 9,925,538 granted 27 Mar. 2018; U.S. Pat. No. 10,509,022 granted 17 Dec. 2019; U.S. Pat. No. 10,533,229 granted 14 Jan. 2020; U.S. Pat. No. 10,350,601 granted 16 Jul. 2019; U.S. Pat. No. 10,449,543 granted 22 Oct. 2019; U.S. Pat. No. 10,466,160 granted 5 Nov. 2019; U.S. Pat. No. 10,391,493 granted 27 Aug. 2019, U.S. Pat. No. 10,633,693 granted 28 Apr. 2020; and U.S. application Ser. No. 16/816,817, filed 12 Mar. 2020, which are each incorporated in their entirety by this reference.

Embodiments, variations, and examples of compositions of particles are described in Section 3 below, and in U.S. application Ser. No. 17/109,704 filed on 2 Dec. 2020, which is herein incorporated in its entirety by this reference.

### 2.1 Method-Distributing Functionalized Particles Across Partitions

Step S110 recites: distributing a set of functionalized particles, in a first morphological state (e.g., a first state of swelling), across a set of partitions using a first medium (e.g., during reconstitution of functionalized particles), which functions to load the functionalized particles into partitions and/or onto a substrate in an efficient manner.

The set of partitions can include wells (e.g., microwells, nanowells) distributed across a substrate (e.g., plate, cartridge, chip, slide, etc.), where such wells can include a base surface, one or more walls extending from the base surface to define a chamber volume, and an open surface (which can be closed or other wise covered during stages of use). In variations, the chamber volume can have a length, a width, and a depth, where the length (or diameter) can vary from 1-50 microns, 1-60 microns, 1-70 microns, or greater. The width (or diameter) can vary from 1-50 microns, 1-60 microns, 1-70 microns, or greater. The depth can vary from 1-80 microns, 1-90 microns, 1-100 microns, 1-200 microns, or deeper; however, in other variations, the chamber volume can have other suitable ranges of characteristic dimensions. Additionally or alternatively, thicknesses of walls separating adjacent wells of the set of partitions can be 0.1 to 1 microns thick, 0.1 to 2 microns thick, 0.1 to 3 microns thick, or another suitable thickness. Furthermore, where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention, as described here and throughout this disclosure.

In examples the depth (or other characteristic of the chamber volume) can be configured to accommodate a target material component (e.g., cell, tissue structure, protein, nucleic acid, other analyte) of the sample and a functionalized particle in a morphological state associated with sample processing. Alternatively, the chamber volume can be sized smaller than a combined volume of the target material component and the functionalized particle, in relation to morphology-adjustable particles and morphological states of such particles (e.g., in relation to particle loading modes, particle release modes, etc.).

In more detail with respect to different variations, in one use case, hydrated particles (e.g., particles in a third morphological state) are in a third state of swelling and have diameters larger in diameter than the microwell diameter of the set of partitions. By tuning size, particles hydrated with a first medium and then shrunk (e.g., with use of a medium, with dehydration, etc.), may shrink to the point where they enter the microwell. The shrunken particle size may be such that only one particle can fit within microwell. As such, distributing particles in a swollen state, followed by shrinking, can improve the efficiency of having a 1:1 ratio of particles to wells, without needing redistribution of particles (e.g., with flow to dislodge any additional particles beyond a first particle within a well). In another use case, the height of the microwell is identical to the height of the loaded particle in a first morphological state. Method steps involving shrinking or further shrinking the loaded particle (e.g., with a second medium providing an osmotic shift, with dehydration, etc.) to a second morphological state can provide a volume for the loading of a substrate (e.g., a cell) within the well given the increased well capacity for additional contents due to the second morphological state of the particle. As such, the second morphological state can be a second state of swelling, in which the set of functionalized particles are dehydrated and have a second size smaller than the first size of particles in the first morphological state.

In another use case, the particle is designed to dissolve upon the addition of an exogenous agent. As such, variations of the method 100 can include transitioning the set of functionalized particles to a fourth morphological state in which the set of functionalized particles are dissolved with an exogenous agent. In one example, the particle can comprise N, N′-Bis(acryloyl)cystamine, which dissolves when reacted with reducing agents such as DTT or TCEP. In the use case where a dissolvable hydrogel is used, the particle can be dried after loading. When loading a substrate such as a cell, the dissolving agent can be added such that upon rehydration, the particle dissolves and occupies no or neglible space of the respective well, thus allowing for the occupancy of the microwell by the substrate (e.g., a cell).

In examples where the target material component is a cell, the chamber volume can have a height or other characteristic that is as tall or taller than a combined height of the cell and the functionalized particle (e.g., in a reconstituted state). Alternatively, the chamber volume can have a height or other characteristic that is less than (e.g., half, less than half, less than by another factor) a combined height of the cell and the functionalized particle. Examples of use and sample processing in which the chamber volume has a height or other characteristic that is less than a combined height of the cell and the functionalized particle can thus be implemented, in relation to initial delivery, drying, and reconstitution of functionalized particles, and processing of functionalized particles with target sample material. For instance, equilibrium states of functionalized particles can be tuned, on-demand, in a manner that produces particle sizes that remain stably smaller than their maximum swollen sizes, to allow for use of microwells having heights smaller than a combined height of a single cell and a single full-sized functionalized particle. In another example, shrunken and/or dried particles can be dissolved when loading the cell such that the particle no longer occupies volume in the partition (e.g., microwell), thereby enabling loading of the cell and/or preventing displacement of the cell by the particle during cell loading. Similarly, the particles can be deliberately sized and/or shaped to allow loading of only 1 target (e.g., 1 cell or 1 nucleus) per partition at loading, but then optionally shrunken or dissolved for subsequent steps; particles may be either smaller, larger, or the same size in prior or later workflow steps. By use of microwells having smaller heights, overloading of microwells with cells, functionalized particles, and/or other materials can be avoided, enabling loading of the partition (e.g., microwell) with only one cell (or other target) and/or functionalized particle.

Distributing the set of functionalized particles can be performed using a fluid handling system with liquid aspiration and delivery components, involving automated distribution apparatuses, where embodiments, variations, and examples of components are described in Patent Applications, Publications, and Issued Patents incorporated by reference. Additionally or alternatively, distributing the set of functionalized particles can be performed manually, with fluid delivery components (e.g., pumps, syringes, etc.) structured for handling of functionalized particles (e.g., in solution, in another medium).

Embodiments, variations, and examples of well systems and processing platforms are described in one or more of: U.S. Publication number 2018/0364148 filed 27 Jul. 2018; U.S. Publication number 2019/0144931 filed 30 Jul. 2018; U.S. Pat. No. 9,925,538 granted 27 Mar. 2018; U.S. Pat. No. 10,509,022 granted 17 Dec. 2019; U.S. Pat. No. 10,533,229 granted 14 Jan. 2020; U.S. Pat. No. 10,350,601 granted 16 Jul. 2019; U.S. Pat. No. 10,449,543 granted 22 Oct. 2019; U.S. Pat. No. 10,466,160 granted 5 Nov. 2019; U.S. Pat. No. 10,391,493 granted 27 Aug. 2019, U.S. Pat. No. 10,633,693 granted 28 Apr. 2020; and U.S. application Ser. No. 16/816,817, filed 12 Mar. 2020, which are each incorporated in their entirety by reference above.

While wells are described, distribution in Step S110 can be performed across surfaces or within partitions not involving wells. For instance, the set of particles can be distributed across recesses (e.g., dimples) on a substrate, between protrusions (e.g., bumps) on a substrate, as spots or droplets on a substrate (e.g., with markers or fiducials to provide separation), within droplets, and/or in another suitable format. As such, the distribution can be in a pattern (e.g., governed by an array of recesses or wells) and/or in a packed configuration (e.g., hexagonal close packed configuration, other close packed configuration). Additionally or alternatively, the distribution can be non-uniform and/or in a non-packed configuration.

Distribution in Step S110 can also occur using a sol-gel solution such that when tunable particles in the sol-gel solution are distributed, the sol-gel solution is solidified. In some cases, the solidified sol-gel matrix is not sensitive to the particle size tuning agents. For instance, distributing the set of functionalized particles can be performed by printing particles (e.g., in droplet format) using a liquid ink composition of particle material, where, upon deposition at a surface or within microwells, the liquid ink can be cross-linked to form solid bodies of the functionalized particles.

Distribution of the set of functionalized particles can include one or more of: priming the substrate (e.g., wells of the substrate) or container associated with the partitions, incubating the set of functionalized particles to prepare them for distribution and/or for storage, washing the set of functionalized particles (e.g., in one or more stages), aliquoting a desired number or volume of functionalized particles for distribution across partitions, constituting or reconstituting (e.g., from a non-constituted state) the set of functionalized particles using a first medium, depositing the set of functionalized particles across the partitions of the substrate or container, and/or performing other suitable steps. Preparation and distribution can be performed using apparatuses as described, and/or using deposition apparatus, some variations of which are described further below.

In variations, priming can include priming in ethanol (e.g., 100% ethanol, 90% ethanol, 70% ethanol, etc.) and/or another suitable priming medium, in order to prepare the substrate or container(s) for receiving the distribution of functionalized particles.

In variations, incubating can include incubating the set of functionalized particles in an incubation medium (e.g., water, another suitable medium) at a desired temperature (e.g., from 10-37° C., at another suitable temperature or temperature profiles), and for a desired duration of time (e.g., from 1-90 minutes, for less than 1 minute, for longer than 90 minutes); however, in other variations, incubation can be performed in another suitable manner.

In variations, washing can include washing the distribution of functionalized particles in water with other additive components (e.g., pluronic F-98, salts, other suitable solutes, etc.), in one or more stages, and with removal of wash media in one or more stages. In examples, washing can be performed from 1 to 5 times, with removal of wash media between washes; however, washing can be performed any other suitable number of times. Additive components can be provided at a percentage of 0.05 to 1%, or another suitable percentage of wash media.

In variations, constituting or reconstituting in the first medium can include constituting/reconstituting in one or more of: water, saline (e.g., phosphate buffered saline, other saline), and/or other buffers. Reconstitution media can include an aqueous media (e.g., water, saline, phosphate buffered saline). Alternatively, reconstitution media can include non-aqueous components. Additionally or alternatively, constituting/reconstituting can be performed using media including polymer components that control the first morphological state of particles of the set of functionalized particles. For instance, reconstitution media can include media tuned to have a suitable density, viscosity, polymer size, polymer branching, refractive index, surfactant properties, or other suitable characteristic to control the first morphological state of particles of the set of functionalized particles in association with the initial distribution step. In examples, additional components affecting properties of reconstitution media can include one or more of: lauric acids, sorbitans, dimethicones, silicone polyether copolymers, hydrogenated polyisobutylene, lipophilic materials, polyethylene glycols (PEGs), iodixanols, polysaccharides, colloidal silica and/or other suitable components. In one variation, the first medium and second medium can each include an aqueous medium, and a process fluid of the set of process fluids used can include a polymer component configured to counteract osmotic swelling of the set of functionalized particles.

Initial reconstitution can produce particles that, in the first state, have a characteristic dimension from 0.5 to 100 micrometers in diameter. However, initial reconstitution can produce particles having other characteristic dimensions and/or other morphologies (e.g., non-spherical morphologies, tube morphologies, etc.). Furthermore, reconstitution can be performed in stages, such that the first medium includes multiple components that are delivered in sequence, to control arrival of the set of particles at the first morphological state. In one such variation, a first component of the first medium can prime the set of functionalized particles for exposure to a second component (and/or third component, fourth component, etc.) of the first medium, to produce particles that are characterized by the first morphological state.

In variations, depositing can include flowing a solution of reconstituted particles over partitions at a substrate, and centrifuging the substrate with the solution of functionalized particles to uniformly distribute the functionalized particles into the partitions. In other variations, depositing can include one or more of: printing functionalized particles, drop-casting solutions of functionalized particles, impregnating (e.g., impregnating droplets with functionalized particles), pipetting functionalized particles as spots on a substrate, promoting gravity settling of functionalized particles into partitions, applying a magnetic field having a characteristic field pattern for attracting magnetic functionalized particles, applying an electric field having a characteristic field pattern for attracting magnetic functionalized particles or charged particles, and any other suitable deposition process. Additionally or alternatively, where partitions are not pre-defined by structures (e.g., as in microwell chips), depositing can include patterning a coupling layer (e.g., an adhesive layer) onto a base substrate (e.g., slide) by a process (e.g., a spraying process, a vapor deposition process, a spin-coating process, a printing process, etc.) to define regions intended for functionalized particles. Thereafter, depositing the functionalized particles onto the prepared substrate can include variations techniques described above, whereby the particles bond (in a permanent manner, in a reversable manner) with the regions of the coupling layer. In variations, where the particles are intended to be removable from a coupling layer, removal can be achieved with a suitable detergent or another reagent/mechanism.

Embodiments, variations, and examples of depositing particles are further described in patent applications, patent publications, and issued patents incorporated by reference above, as well as in U.S. Pat. No. 10,391,492 issued 27 Aug, 2019 and U.S. Pat. No. 10,633,693 issued 28 Apr. 2020, which are each incorporated in its entirety herein by this reference.

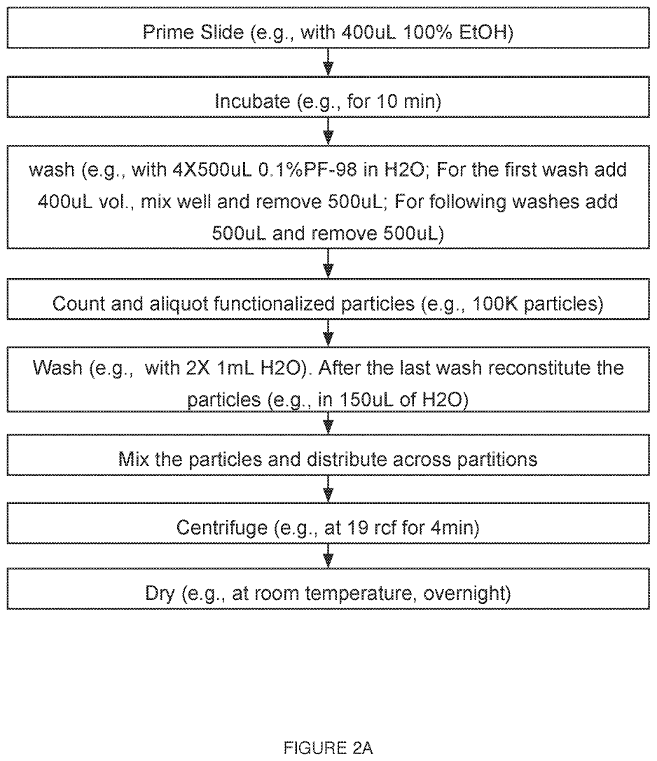

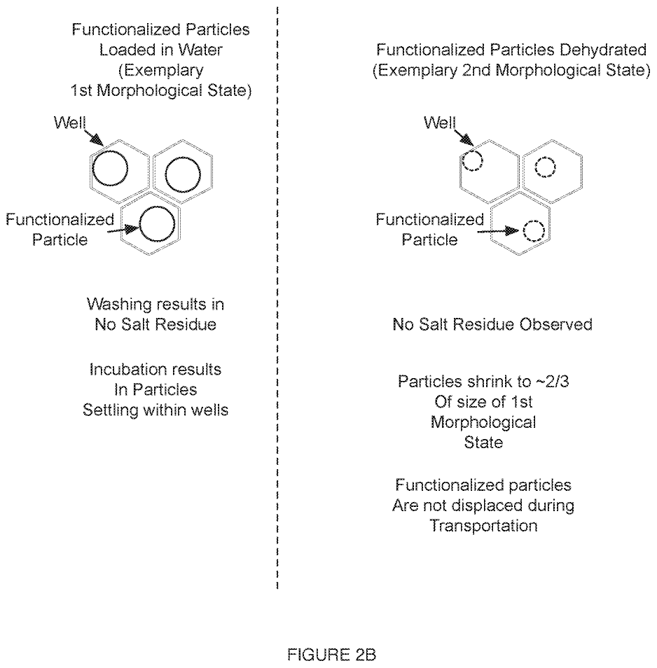

A specific example of step S110, as shown in FIGS. 2A and 2B, can include: priming a substrate having an array of microwells with a volume (e.g., 400 microliters, another suitable volume) of ethanol (e.g., 100% ethanol), followed by incubation for 10 minutes, followed by four stages of washing with a volume (e.g., 500 microliters, another suitable volume) of 0.1%PF-98 in water, with removal of wash media between washes. As shown in FIGS. 2A and 2B, the specific example can further include aliquoting a desired number (e.g., 100K, another suitable number) of functionalized particles, washing the number of functionalized particles (e.g., twice in 1 milliliter of water, another suitable number of times in another volume of wash media), followed by reconstitution in a reconstitution medium (e.g., in 150 microliters of water). In the specific example, deposition can include implementation of a fluid handling system to deliver the reconstituted functionalized particles onto a substrate defining the array of microwells, followed by centrifugation at 19 relative centrifugal force (rcf) for 4 minutes. The example of step S110 produces reconstituted functionalized particles that are loaded into the microwells of the substrate in a uniform manner and without exhibiting salt residues.

Variations of the exemplary method described above can alternatively include priming the substrate having the array of microwells with less than 200 microliters, less than 300 microliters, less than 400 less than 500 microliters, less than 600 microliters, an intermediate volume, or a greater volume of priming solution. Variations of the exemplary method described above can alternatively include priming the substrate having the array of microwells with less than 100% ethanol. Variations of the exemplary method described above can include incubating the substrate for less than 5 minutes, less than 10 minutes, less than 15 minutes, or less than 20 minutes. Variations of the exemplary method described above can include less than 2 stages of washing, less than three stages of washing, less than 4 stages of washing, less than 5 stages of washing, or other washing protocols as necessary, with other constitutions of wash media (e.g., other percentages of PF-98) or other wash media. Variations of the exemplary method described above can include aliquoting 50K, 100K, 200K, 300 400K, 500K, 600K, 700K, 900K, 1M, 2M, 3M, 4M, 5M, 6M, 7M, 8M, 9M, 10M, an intermediate number of functionalized particles, or another suitable number of functionalized particles (e.g., depending upon the number of microwells available at the substrate). Variations of the exemplary method described above can include washing and/or reconstituting the set of functionalized particles in another suitable volume of reconstitution medium. Variations of the exemplary method described above, with respect to deposition, can include implementation of a fluid handling system, printing system, other robotic apparatus, magnetic field, or other suitable mechanism to deliver the reconstituted functionalized particles onto a substrate defining the array of microwells. Variations of the exemplary method described above can include centrifugation at 14 rcf, 15 rcf, 16 rcf, 17 rcf, 18 rcf, 19 rcf, 20 rcf, or another suitable level of rcf, and/or at a duration of 2 minutes, 3 minutes, 4 minutes, 5 minutes, 10 minutes, 15 minutes, etc to facilitate positioning of the functionalized particles within microwells.

In variations involving controlled distribution of the set of functionalized particles within microwells (e.g., with printing, with pick-and-place operations, etc.), variations of the methods described can achieve another desired controlled distribution of particles to wells (e.g., less than 1 particle per well on average, 2 particles per well, 3 particles per well, etc.). Alternatively, the methods can be used to pre-load microwell substrates with particles in a desired pattern. For instance, the microwell substrate can be pre-loaded with a first particle type (e.g., configured to process a first type of target of a sample) in a first pattern or at a first subregion of the microwell substrate, and with a second particle type (e.g., configured to process a second type of target of the sample) in a second pattern or at a first subregion of the microwell substrate, such that distinct target types of the sample can be differentially captured, processed, and/or detected based upon the predetermined first pattern, second pattern, first subregion, or second subregion of the microwell substrate. The first subregion, second subregion, third region, and/or additional subregions can be ordered or otherwise random. In downstream applications, where the different subregions have functionalized particles with varied morphological states and/or varied transition mechanisms for different equilibrium morphological states, method steps can involve controlled staging of particle morphology transitions in relation to an assay being performed. For instance, where a first particle subtype changes (e.g., swells, shrinks, dissolves, folds, etc.) under first conditions and a second particle subtype changes (e.g., swells, shrinks, dissolves, folds, etc.) under second conditions, the method can include transitioning the first particle subtype to its second morphological state (e.g., involving release of the first particle subtype with a first set of captured targets of, for instance, a first cell type) from microwells with a high degree of selective efficiency (e.g., greater than 90%, greater than 95%, greater than 99%, etc.), followed by transitioning the second particle subtype to its second morphological state (e.g., involving release of the second particle subtype with a second set of captured targets for instance, of a second cell type) from microwells with a high degree of selective efficiency (e.g., greater than 90%, greater than 95%, greater than 99%, etc.).

However, variations of the specific example of step S110 can be implemented in another suitable manner.

### 2.2 Method-Transitioning Functionalized Particles to a Second Morphological State

Step S120 recites: transitioning the set of functionalized particles, at the set of partitions, from the first morphological state to a second morphological state. The second morphological state can be a state in which particles of the set of functionalized particles are stabilized within the set of partitions (e.g., for storage, to produce desired shelf life characteristics, for shipment, for other movement or transportation, etc.). In particular, stabilization of the set of functionalized particles can retain positions of the set of functionalized particles within the partitions, even when a substrate or container associated with the set of partitions is transported. As described above, where multiple particle subtypes with different available morphological state transitions are involved, Step S120 can involve transitioning each subtype of the set of functionalized particles, at their respective regions of the set of partitions, to their respective second morphological states.

Additionally or alternatively, the second (or any later morphological state) can be a state in which structure or structural integrity of the set of functionalized particles is altered, such that reconstitution or re-hydration during use of the set of functionalized particles produces a third morphological state (as described in relation to Step S130 below) that is different than the first morphological state, even when using the same media for constitution/reconstitution as in Step S110. In examples, this was initially achieved as a beneficial byproduct of drying and rehydration, but similar results can be achieved by modification of the particles and/or by other suitable means. For example, particles composed of acrylamide monomers crosslinked with both bis acrylamide and BAC (N,N′-Bis(acryloyl)cystamine) will have reduced crosslinking after treatment with a reducing agent, and therefore can show different swelling properties in the same buffer system after reduction of the disulfide linkages. As such, treatment of particles in a first morphological state, with a reagent that adjusts or eliminates all or a portion of crosslinking of bodies the functionalized particles, can be used to produce a a third morphological state (as described in relation to Step S130 below) that is different than the first morphological state.

Additionally or alternatively, the bottom, (e.g., base surface), of a microwell can be configured to couple with a particle such that in all downstream morphological states, the particle remains loaded in the microwell. This attachment can be irreversible or reversible (e.g., using magnetic particles, using particles bonded in a manner that can be reversed with a detergent, using particles bonded in a manner that can be changed in response to an electric field, using particles bonded in a manner that can be changed in response to temperature, etc.).

Additionally or alternatively, the second morphological state can be a state in which the morphology of the set of functionalized particles is otherwise altered (e.g., with particle shrinking, with particle expansion, with structural folding, with inducing a shape memory state, etc.).

In one variation, transitioning the set of functionalized particles to the second morphological state can include drying the set of functionalized particles, within the set of partitions, where drying can be performed passively and/or actively. Passive drying can include drying within a drying environment (e.g., sterile environment, room temperature environment, etc.) without active heating of the set of functionalized particles, partitions, or associated substrates. Active drying can include drying within an altered drying environment having one or more of: a desired gas composition, a desired/low level of humidity, a set temperature (e.g., higher than room temperature), with provision of desiccants, providing convective heating, providing radiative heating, providing active sterilization (e.g., by heat sterilization, by flash sterilization, by ethylene oxide sterilization, by plasma sterilization, etc.), and/or other suitable characteristics. Additionally, active drying can include solvent exchange where aqueous solution is replaced with a non-aqueous solution whether in a single step or multiple steps (e.g., an alcohol dehydration series).

In variations, particles can be dried without any excipients (e.g., in deionized water) and in other variations, the particles may be dried in the presence of excipients which may provide beneficial attributes including: stability of the particles within the partitions (e.g., coupling the particles to partition/well surfaces), desired properties upon rehydration (e.g., with use of PEG or other reagents to control particle size), or desired properties in downstream steps or stability of the particles or associated molecules.

Transitioning the set of functionalized particles to the second morphological state can additionally or alternatively include affecting structure of the set of functionalized particles in another manner. For instance, transitioning in Step S120 can include one or more of: inducing crosslinking (e.g., by application of heat, by application of pressure, by inducing changes in pH, by photo crosslinking, by chemical crosslinking, etc.), removing crosslinks (e.g., by application of heat, by inducing changes in pH, by irradiation, by changing a chemical environment, by mechanical disruption, etc.), reducing structural integrity (e.g., with an acid, with a solvent, by freezing, by thawing, by repeated freeze-thaw cycles, by drying, by chemical reduction of crosslinkers, etc.), and/or affecting structure in another suitable manner.

In an example, as shown in FIG. 2B, dehydration of the set of functionalized particles within partitions, at room temperature for approximately 6-10 hours, produces particles that are ˜⅔ the size of that of the first morphological state, that exhibit no salt residue, and that retain their positions within partitions, even when the substrate defining the well partitions is moved or transported. In variations of the exemplary method described, dehydration of the set of functionalized particles within partitions, can occur at temperatures warmer than room temperature, for less than 6 hours or greater than 10 hours, in order to achieve particles that are another suitable size in relation to sizes of particles in the first morphological state. However, variations of the example can be performed in another suitable manner.

2.3 Method—On-Demand Tuning of Morphological States of Functionalized Particles, in Coordination with Target Capture and Processing

Step S130 recites: processing the set of partitions with one or more of a set of process fluids, thereby transitioning the set of functionalized particles, at the set of partitions, from the second morphological state to a third morphological state (e.g., third state of swelling). Step S130 functions to transition the set of functionalized particles, within their respective partitions, to a state ready to be used in sample processing for target capture, material synthesis, and/or characterization. Step S130 can additionally or alternatively function to provide a non-intuitive mechanism for releasing functionalized particles from their respective partitions on demand and/or for retrieval of target material captured from the sample when using the set of functionalized particles.

In variations, the third morphological state can be reconstituted state, hydrated state (e.g., in aqueous media), state associated with shape memory characteristics of the functionalized particle (e.g., due to environmental pH, due to environmental heat, due to environmental pressure, due to applied electric fields, due to aspects of surrounding media, etc.), a partially degraded state (e.g., such that a larger degree of swelling can occur, in comparison to the first morphological state), a fully degraded state (e.g., with compromise of structural integrity to release material, such as primers or other components attached to the particle matrix), or other state (e.g., in aqueous and/or non-aqueous media). The third morphological state can thus return functionalized particles to their same state (e.g., size, shape, etc.) as the first morphological state in Step S110, or a different state (e.g., size, shape, etc.) as the first morphological state in Step S110.

Non-intuitively, in examples, use of the same media (e.g., such that the first medium is identical to the second medium) used to constitute/reconstitute the functionalized particles in Step S110 transitions the functionalized particles to have a larger equilibrium size (i.e., as the third morphological state) than that of the first morphological state. As such, the third morphological state can be a third state of swelling in which the set of functionalized particles have a third size greater than the first size. Such properties can thus provide an on-demand mechanism for releasing functionalized particles, with or without associated material from a sample, from partitions for downstream retrieval and further applications. Furthermore, when different particle subtypes are involved, and each particle subtype can be individually released with a triggering stimulus (e.g., media change, etc.) such a configuration can provide an on-demand mechanism for releasing subsets of functionalized particles (e.g., with different captured targets) sequentially or in stages, with a high degree of efficiency, as described above. As further described below, use of various media and/or environmental conditions can otherwise produce different equilibrium particle states as the third morphological state. Additional properties and applications are further described below.

Transitioning the set of functionalized particles to the third morphological state can include one or more of: priming the substrate (e.g., wells of the substrate) or container associated with the partitions, incubating the set of functionalized particles to prepare them for distribution and/or for storage, washing the set of functionalized particles (e.g., in one or more stages, with one or more different types of wash media), providing various environmental conditions for the set of functionalized particles in coordination with stages of sample processing, and/or performing other suitable steps. Delivery or reception of different process fluids of the set of process fluids can be performed using apparatuses as described, and/or using deposition apparatus, some variations of which are described further below.

In variations, priming can include priming in ethanol (e.g., 100% ethanol, 90% ethanol, 70% ethanol, etc.) and/or another suitable priming medium, in order to prepare the substrate or container(s) for receiving the distribution of functionalized particles.

In variations, incubating can include incubating the set of functionalized particles in an incubation medium (e.g., water, another suitable medium) at a desired temperature (e.g., from 10-37° C., at another suitable temperature or temperature profiles), and for a desired duration of time (e.g., from 1-90 minutes, for less than 1 minute, for longer than 90 minutes); however, in other variations, incubation can be performed in another suitable manner. Incubation can additionally or alternatively be performed in coordination with incubation steps (e.g., incubation related to cell lysis, incubation related to cell culture, incubation related to enzymatic processes, etc.) for an assay being performed with the set of functionalized particles.

In variations, washing can include washing the distribution of functionalized particles in water with other additive components (e.g., polyethylene glycol, other polymer solutions, salts, other suitable solutes, etc.), in one or more stages, and with removal of wash media in one or more stages. In examples, washing can be performed from 1 to 5 times, with removal of wash media between washes, and using mixtures of PEGs in saline (e.g., 15% PEG 6000 in 1X phosphate buffered saline, other mixtures); however, washing can be performed any other suitable number of times. Additive components can be provided at a percentage of 0.05 to 20%, or another suitable percentage of wash media. In particular, washing with media including polymer solutions or other solutions be used to control equilibrium swelling point(s) for the functionalized particles and/or control states in other suitable manners.

Additionally or alternatively, washing media and/or reconstitution media can include non-aqueous components. Additionally or alternatively, washing media can be performed using media including polymer components that control the third morphological state of particles of the set of functionalized particles, by countering osmotic pressure-induced swelling of particles. For instance, media can include components tuned to have a suitable density, viscosity, polymer size (e.g., molecular weight, branching characteristics), polymer branching, surfactant properties, or other suitable characteristic to control the third morphological state of particles of the set of functionalized particles at equilibrium. In examples, additional components affecting properties of reconstitution media can include one or more of: lauric acids, sorbitans, dimethicones, silicone polyether copolymers, hydrogenated polyisobutylene, lipophilic materials, polyethylene glycols (PEGs), iodixanols, polysaccharides, colloidal silica, and/or other suitable components. Additionally or alternatively, salts can be provided with wash and/or reconstitution media, in order to affect charge of the environment surrounding the set of functionalized particles, thereby affecting equilibrium swelling state.

Washing/reconstitution can produce particles that, in the third morphological state, have a characteristic dimension from 0.5 to 100 micrometers in diameter. However, washing/reconstitution can produce particles having other characteristic dimensions and/or other morphologies (e.g., non-spherical morphologies, tube morphologies, morphologies involving split bodies, morphologies involving wrinkled surfaces, morphologies involving larger pore sizes, etc.).

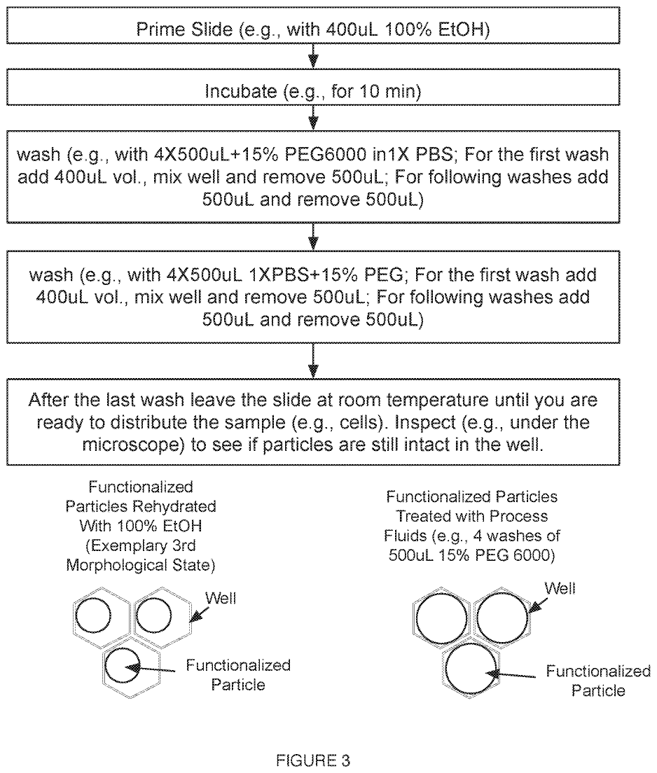

A specific example of step S130, as shown in FIG. 3, can include:
priming the substrate having the functionalized particles, stored in the second morphological state, with a volume (e.g., 400 microliters, another suitable volume) of ethanol (e.g., 100% ethanol), followed by incubation for 10 minutes, followed by multiple stages of washing with a volume (e.g., 500 microliters, another suitable volume) of 15% PEG 6000 in 1X PBS, with removal of wash media between washes. The specific example can be performed multiple times, and/or with variations (e.g., in volume and percentage of ethanol, number of wash steps and wash medium, and application of a stabilizing medium, other variations of which steps are described above). As shown in FIG. 3, the specific example of Step S130 provides functionalized particles, within the set of partitions, that swell to an equilibrium state in which additional sample materials (e.g., cells, nucleic acids, proteins, other material) can be distributed to the partitions, in order to interact with functionalized components of the set of functionalized particles.

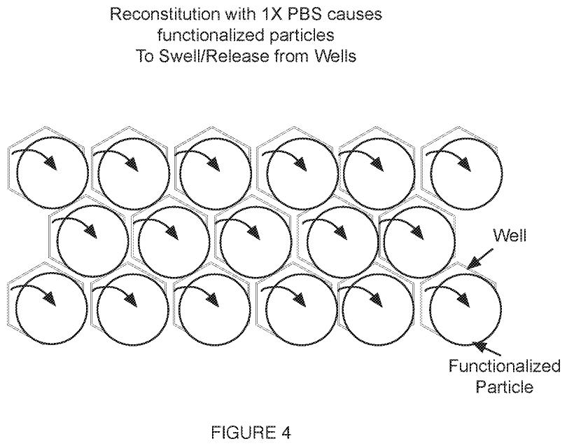

In another specific example, as shown in FIG. 4, reconstitution with saline (e.g., 1X PBS), as used in examples to initially reconstitute functionalized particles in coordination with distribution across partitions, induces swelling of functionalized particles to a larger equilibrium state than the first morphological state, in a non-intuitive manner. As such, the specific example shown in FIG. 4 can provide an on-demand mechanism for swelling and release of functionalized particles from partitions (e.g., such that functionalized particles swell and float away from partitions, for downstream retrieval of functionalized particles, with target materials, for further analysis/processing. In the specific example, functionalized particles that are initially ˜20 micrometers in diameter in PBS swell to 25 micrometers in diameter, upon rehydration with PBS in Step S130 (where PBS was also used as the first medium in earlier stages). Variations of starting and rehydration media can also be implemented, to achieve different degrees of subsequent swelling in comparison to initial states in the same medium. As such, particles can swell to a size (in the third morphological state) that is 15% greater than the first morphological state, 20% greater than the first morphological state, 25% greater than the first morphological state, 30% greater than the first morphological state, 40% greater than the first morphological state, an intermediate percentage, or another percentage greater than the first morphological state.

As such, in variations where the set of partitions include a set of microwells, transitioning the set of functionalized particles to the third morphological state can include releasing the set of functionalized particles from the set of microwells by inducing swelling in response to the second medium (or another medium).

In other examples, reconstitution with applied environmental conditions (e.g., pH conditions, temperature conditions, electric field conditions, pressure conditions, enzymatic conditions to affect cross-linking, irradiation conditions, magnetic field conditions, etc.) can produce third morphological states, states bypassing the third morphological states, or optionally, subsequent morphological states, associated with equilibrium shape memory states under various environmental conditions. Such third morphological states can include folded states, unfolded states, states producing motility, geometric or volumetric configurations, states of reduced structural integrity, and/or other states.

However, embodiments, variations, and examples of Step S130 can be performed in other suitable manners.

Furthermore, in relation to Step S10 and/or other steps associated with various morphological states shown in FIGS. 1A and 1B, different functional modes can be provided, by controlling morphology of the functional particles in an on-demand manner.

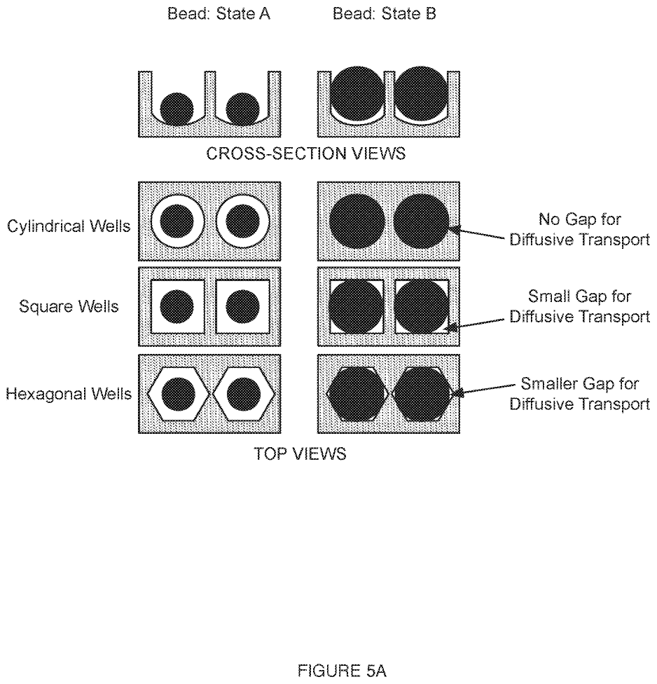

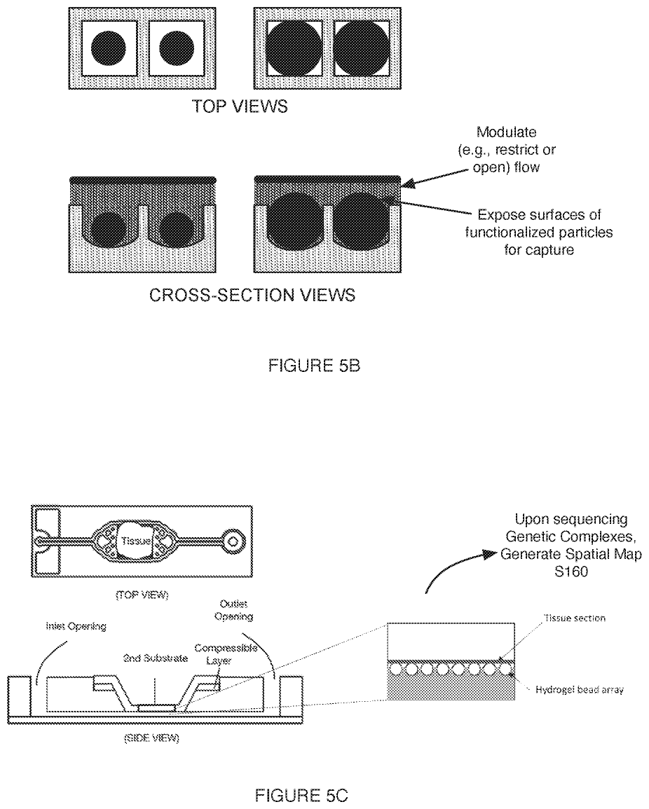

In variations shown in FIG. 5A, the functionalized particles can be positioned within partitions in a second morphological state (e.g., State A shown in FIG. 5A, left), and then transitioned to a third morphological state (e.g., State B shown in FIG. 5B, left). In more detail, functionalized particles in State B are swollen or otherwise expanded (e.g., according to methods described), where partition morphologies (e.g., microwell cross-sectional shapes) can provide: sealing with no gap or opening for diffusive transport (e.g., of reagents) past functionalized particles in State B (e.g., for microwells with open surfaces having a circular cross sections or sections complementary to that of the functionalized particle cross section). As such, transitioning the set of functionalized particles to the third morphological state can include sealing the set of microwells at said open surfaces with the set of functionalized particles in the third morphological state. Partition morphologies (e.g., microwell cross-sectional shapes) can alternatively provide partial sealing with a small gap or opening for diffusive transport (e.g., of reagents) past functionalized particles in State B (e.g., for a microwell with a non-circular/polygonal cross section having 5 or fewer sides); or partial sealing with a smaller gap or opening for diffusive transport (e.g., of reagents) past functionalized particles in State B (e.g., for a microwell with a non-circular/polygonal cross section having 6 or greater sides). In the variations shown in FIG. 5A (right), a higher degree of similarity between a cross section of the functionalized particle to a cross section of the partition/microwell provides a smaller gap for diffusive transport (e.g., of reagents) past functionalized particles in State B. As such, transitioning the set of functionalized particles to the third morphological state can include partially sealing the set of microwells at said open surfaces with the set of functionalized particles in the third morphological state, where partially sealing the set of microwells provides a gap for diffusive transport of reagents past the set of functionalized particles within the set of microwells.

In variations shown in FIG. 5B, transitioning of functionalized particles retained within partitions/microwells from a second morphological state to a third morphological state (e.g., swollen or expanded state) can be used to modulate flow within a channel, cavity, or other structure defining a flow path over the array of partitions (e.g., set of microwells). For instance, controlled particle swelling can restrict flow by reducing flow path cross sectional area, and controlled particle shrinking can open flow by enlarging flow path cross sectional area. Furthermore, controlled swelling and shrinking of functionalized particles can provide mechanisms for controllably or periodically exposing functionalized particle surfaces for capture of molecules/biomarkers transported through the flow path. As such, in variations where the set of partitions include a set of microwells positioned adjacent to a cavity defining a flow path over the set of microwells, and transitioning the set of functionalized particles to the third morphological state can include restricting flow (or controllably exposing particles to components) within the cavity by decreasing a cross section of the flow path, upon inducing swelling of the set of functionalized particles in response to the second medium (or another medium).

In variations shown in FIG. 5C, transitioning of functionalized particles to another morphological state (e.g., swollen or expanded state) can provide improved surface contact area (e.g., with hydrogel beads) between functionalized particles and a tissue sample, in order to characterize distributions of targets in the tissue sample. For instance, controlled states of swelling of the set of functionalized particles from addressable microwells can be used in the context of spatial transcriptomics, where, decoding positions of functionalized particles and captured targets (e.g., through sequencing) can be used to create spatial maps of captured targets. In one exemplary use case, particles can be loaded within microwells in a shrunken state, transitioned to a state in which they protrude from their respective microwells (or are otherwise able to contact sample material) by hydration with desired osmolarity factors, and interacting the microwell array, with protruding functionalized particles, with a sample (e.g., tissue sample) with the particles can be used to determine relative spatial positions of targets of the tissue sample (e.g., single cells, single cell subtypes, proteins, nucleic acids such as RNAs and DNAs, other biomarkers).

As such, in variations where the sample is a tissue sample and the set of partitions includes a set of microwells, the method 100 can include inducing contact between the tissue sample and the set of functionalized particles, upon transitioning the set of functionalized particles to the third morphological state in which the set of functionalized particles protrude from open surfaces of the set of microwells, by inducing swelling the set of functionalized particles in response to the second medium (or another medium). Upon sequencing genetic complexes resulting from said interactions between the set of functionalized particles and the tissue sample, the method 100 can then include generating a spatial map of the set of targets, thereby characterizing relative spatial distributions and/or positions of targets within the tissue sample.

In variations, the tissue sample can include one or more of: a whole tissue, a tissue portion (e.g., a histological tissue slice), a formalin-fixed paraffin-embedded (FFPE) tissue, a frozen tissue, a biopsied tissue, fresh frozen plasma, a cell-seeded scaffold, an analyte-seeded, an organ, a whole organism, an organoid, a cell suspensions including a single cell type or mixture of cells within a medium, a suspension of nuclei, an organelle, or other sample.

Furthermore, transitions between states of the functionalized particles can be used to transfer functionalized molecules (e.g., probes) between the functionalized particles and the tissue sample to enable downstream analyses according to various assays.

Furthermore, controlled degradation of the functionalized particles can facilitate release of genetic complexes generated using the set of functionalized particles, for downstream processes.

The compositions can further provide other controlled morphological states to enable other operation modes.

### 2.3.1 Sample Processing, Target Detection, Characterization

In variations, the method 100 can further include Step S140, which recites: inducing interactions between the set of functionalized particles and a set of targets, within the set of partitions and according to a set of operations. Step S140 can include processing one or more samples, using the set of functionalized particles at the partitions, for various tissue, cell, and/or molecular reactions (e.g., involving target capture, synthesis of nucleic acid material, amplification, library preparation, etc.). As such, Step S140 can include distribution of sample solutions and/or processing reagents across the partitions (e.g., using liquid handling apparatus described).

In examples, sample materials can include or be derived from: tissue structures, tissue pieces (e.g., tissue slices for histological applications, biopsied tissues, tissue blocks associated with seeded scaffolds, etc.), droplets of sample material, organs, whole organisms (e.g., bacteria, viruses), seeds, consumable materials (e.g., foods, beverages, pharmaceuticals, supplements etc.), cell suspensions, single cells, organelles, within organelles, crosslinked co-localized histones with attached DNA, exosomes, other microorganisms, and/or other natural or synthetic structures. Targets can thus include cells, cell-derived material, nucleic acids (e.g., RNAs, DNAs, all poly(A) mRNA molecules from a single cell, all DNA molecules from a single cell, other nucleic acids or fragments, etc.), proteins (e.g., all proteins from a single cell), amino acids, analytes, and/or other targets. Further descriptions of targets are described in Patent Applications and Issued Patents incorporated by reference.

In relation to applications of use related to detection and processing of such targets, exemplary applications of use can support one or more of: single cell processing and detection (of single cells, of single cell subtypes, etc.); cancer diagnostics (e.g., in relation to SNP analyses for specific alleles, in relation to assessment of minimum residual disease, in relation to detection of circulating tumor cells, etc.); prenatal testing (e.g., for assessment of chromosomal issues, such as aneuploidy); microbiome analysis (e.g., for characterization of hypervariable regions of 16S rRNA and/or ITS rRNA); whole genome amplification; characterization of genetically modified organisms (e.g., in the context of agriculture, in the context of seed analysis, etc.); and/or other forms of genetic testing.

Processes and reactions can be associated with one or more molecular reactions including one or more of: a reverse transcription reaction (RT-reaction), a cDNA synthesis operation, immunochemistry, DNA reactions, mRNA FISH reactions, proximity ligation reactions, bridge amplification reactions, catalytic enzymatic reactions, hybridization reactions, restriction digestion reactions, amplification reactions (e.g., mRNA and/or DNA PCR, quantitative PCR, digital PCR, isothermal amplification, such as loop-mediated isothermal amplification), and other suitable reactions.

Post-retrieval of genetic complexes using the set of functionalized particles, characterizations can be performed using a sequencing operation, such as: sequencing by hybridization, sequencing by ligation, pyrosequencing, sequencing by synthesis, high-throughput sequencing, and/or other methods. Detection can additionally or alternatively involve fluorescent detection (e.g., involving detection of fluorophores of probes used to tag targets, detection of fluorophores that operate with Foerster resonance energy transfer, etc.).

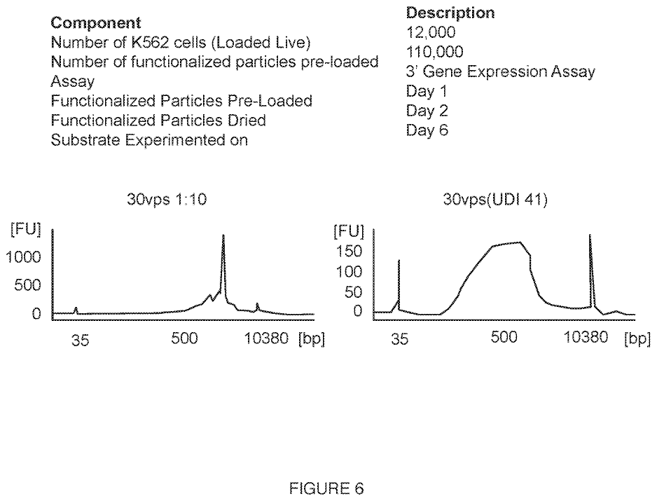

In one example, as shown in FIG. 6, exemplary functionalized particles as described were used to process cells (e.g., 12,000 K562 cells loaded in a viable state), with functionalized particles (e.g., 110,000 particles) pre-loaded across microwell partitions, with performance of a gene expression assay, with capturing of mRNA from cells within the partitions, synthesis of cDNA, and successful generation of an indexed library.

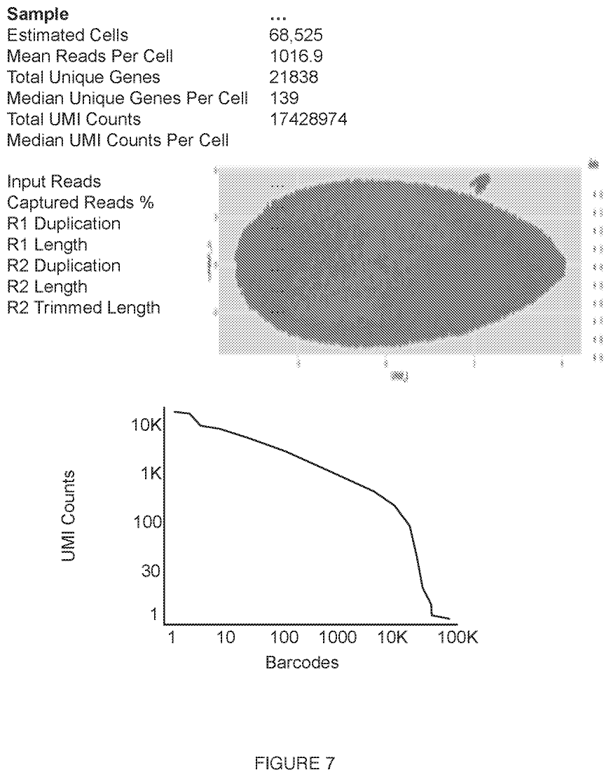

Sequencing results are provided in FIG. 7, demonstrating functionality of particles for single cell processing and sequencing applications, post-drying and storage (e.g., after 5 days, after more than 5 days).

Other embodiments, variations, and examples of assays that can be performed using such functionalized particles within partitions are described in U.S. application Ser. No. 16/890,417 filed 2 Jun. 2020, U.S. application Ser. No. 17/109,704 filed 2 Dec. 2020, U.S. application Ser. No. 17/499,454 filed 12 Oct. 2021, and U.S. application Ser. No. 17/499,529 filed 12 Oct. 2021, which are each incorporated herein in its entirety by this reference, as well as other applications incorporated by reference.

### 3. Functional Bead Composition

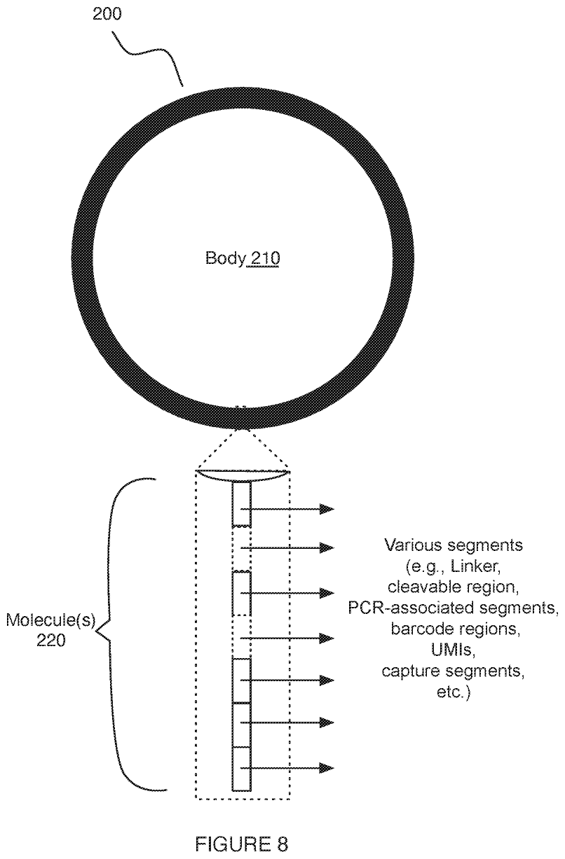

As shown in FIG. 8, an embodiment of a composition 200 for target material separation includes: a body 210 and one or more molecules 220 coupled to the body 110 and structured for functionalization of the composition 200.

In relation to morphology, the body 210 can have the form of a microsphere. Alternatively, the body 210 can have the form of a non-spherical (e.g., ellipsoidal, prismatic, polyhedral, amorphous, etc.) body, where a cross section taken through the body 110 is non-circular. The body 210 can be composed of a polymer (e.g., hydrogel), with functionality as described above. Variations of polymer compositions can include one or more of: polyacrylamides, polystyrenes, silicas, polymethylmethacrylates, aliginate, PEG, nylons, agaroses, and/or other polymers, with or without crosslinking.

Embodiments, variations, and examples of compositions of functionalized particles, with respect to thermal properties, optical properties, composite structures, mechanical properties, magnetic properties, densities, electric properties, physical properties, porosity, crosslinking, functionalization, structures of functionalized molecules, synthesis, and other aspects are further described in U.S. application Ser. No. 17/109,704 filed 2 Dec. 2020, which is herein incorporated in its entirety by this reference.

Additionally or alternatively, functionalized particles can be configured as vessels or vesicles configured to deliver contents to partitions in association with sample processing. For instance, the functionalized particles can be structured with or as semi-permeable membranes to deliver “packets” of materials to partitions (e.g., in microwell systems, in other partition formats). In one example, such functionalized particles can deliver barcoded oligonucleotides to partitions for single cell RNA sequencing applications and/or other applications, in a manner that provides improvements over compositions that require sealing of adjacent partitions (e.g., using a film or oil), where such sealing presents certain challenges to workflows. In more detail, in some applications, it is desirable to have some materials (e.g., mRNA from a cell) stay within respective partitions and other materials (e.g., a lysis reagent, other process reagents) be able to move into the partition to lyse a cell, and then move out of the partition to not interfere with subsequent steps. Delivery of materials to partitions can additionally or alternatively include inducing controlled degradation of functionalized particles, once they are positioned at their respective microwells, to release packaged materials within their respective microwells for target capture and/or other steps of sample processing.

By utilizing properties of functionalized particle designs described herein, semi-permeable barriers can be provided without the problems associated with placement and attachment of membranes or other films/oils to keep partitions divided. Such a configuration also takes advantage of the instrumentation already designed to enable delivery of functionalized particles to partitions, but with the added advantage that said functionalized particles then act as the semi-permeable barrier.

To be effective, the functionalized particle must be sized appropriately in order to fit with limited clearance around the functionalized particle. In variations, partition geometry (e.g., microwell geometry, droplet size, etc.) and functionalized particle size control can be finely tuned to complement each other. Additionally or alternatively, a set of monodisperse functionalized particles, with pores of defined sizes can operate together as a semi-permeable membrane within a partition. In such variations, hydrogel beads with semi-permeable properties can be implemented, and structured to change size in response to environmental conditions (embodiments, variations, and examples of which are described above).

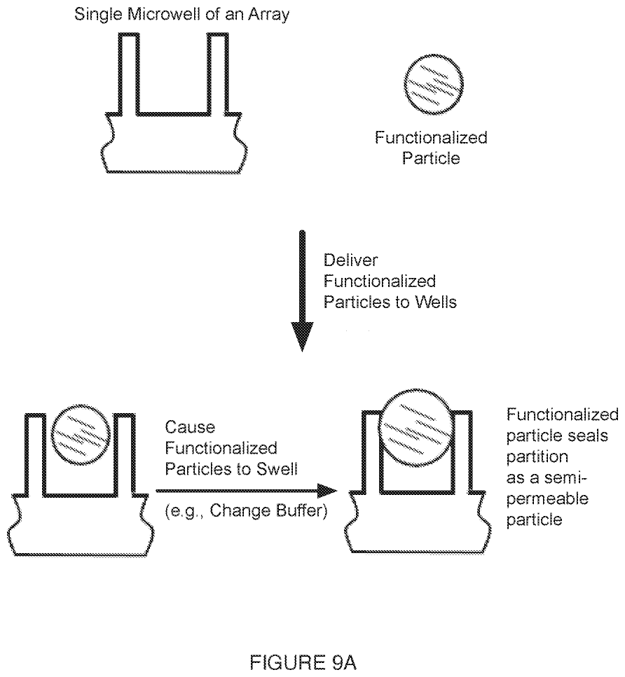

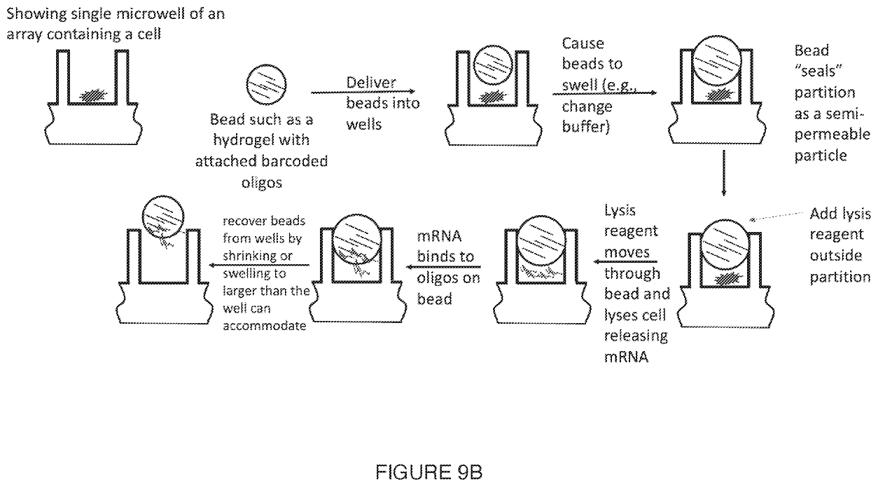

In one such example, a population of hydrogel (e.g., acrylamide crosslinked with bis-acrylamide) functionalized particles (e.g., containing functionalized oligonucleotides linked to the hydrogel matrix) that are sized slightly larger than a microwell opening, can be exposed to buffers (e.g., 10 mM MgCl₂, another salt) containing materials that produce smaller equilibrium sizes of functionalized particles, thereby shrinking the majority of functionalized particles to a size smaller than the microwell opening. Functionalized particles in this state can then be loaded into the microwells, followed by changing the surrounding buffer (e.g., to a lower salt buffer, such as tris EDTA buffer, etc.) to produce swelling but with retention of functionalized particles in the microwells (e.g., as shown in FIG. 9A). Such a process can effectively close off/seal the microwell opening with one or more semi-permeable functionalized particles. Such a structure with tunable morphological states presents only a minor barrier to small molecules such as salts, but presents a barrier to many biomolecules or other larger molecules, thereby preventing leakage of targets. For example, mRNA from a cell would be unlikely to pass through the functionalized particles, whereas salt or certain detergents would be able to pass the functionalized particles (e.g., for buffer exchange, for other purposes).

By loading functionalized particles into partitions, followed by other materials, and then changing the buffer solution in contact with partition contents, there can be small molecule exchange with the partitions (e.g., by diffusion), but larger molecules remain sequestered within the partitions. The specific properties of the functionalized particles can further be tailored to the experimental needs. For example, the pore size of acrylamide can be altered to change the porosity and thus the size of molecules that are able to pass through the matrix. Other hydrogels and non-hydrogel materials could also be used that have specific properties relative to particular molecules of interest. Thus, by taking advantage of the size of the functionalized particles, and in particular the mutability of their sizes, utility is achieved with delivery of functionalized particles to partitions and then transitioning the functionalized particles to states that effectively seal off those partitions without needing the same degree of size control that would be required to precisely match the size of each partition. Such compositions and processes can further be used to achieve self-assembled membranes for isolating partitions that are not uniform (e.g., uniformly cylindrical). For instance, delivery of functionalized particles to wells, and then transitioning them to swollen states such that they seal off their respective wells, can provide a novel mechanism for forming a customizable and self-assembled membrane that can be controlled on-demand.

In an example, microwell partitions implemented included a draft angle to partition walls such that the opening was approximately 27 micrometers in diameter at the open surface and 22 micrometers at the base surface. Upon exchanging of a buffer within microwells to a low salt buffer, functionalized particles constructed of a hydrogel material (e.g., acrylamide) and present in the microwells remained in the microwells as they swelled. As such, the functionalized particles were transitioned to states where they became effective structures to seal partitions in a semi-permeable manner, even though the functionalized particle population has inherent size variation. In this example, functionalized particles that were sized to the opening size or greater were able to operate as semi-permanent membrane to the microwells. As such, the enormous difficulty in manufacturing functionalized particles with the size control that would otherwise be necessary is avoided, and thus by swelling the functionalized particles in situ, it is possible to achieve the desired result in a way that is practical and efficient to achieve.

For recovery, the beads can subsequently be dissolved or changed to a different size (e.g., a smaller size, a larger size, etc.) such that they can be retrieved. As a note, the draft well shape is particularly advantageous for maintaining several sequential states (e.g., a large state that does not fully fit in well, a shrunken state to to fit within and/or seal a well, an expanded state to induce popping of the functionalized particle from a well, etc.).

FIG. 9B depicts an example workflow for single-cell RNA sequencing, where a microwell of an array of microwells contains a single cell (as described in applications incorporated by reference), followed by delivery of a functionalized particle composed of a hydrogel (e.g., acrylamide) with attached barcoded oligonucleotide molecules (as described in applications incorporated by reference) into the microwell. Buffer exchange causes the functionalized particle to swell to an equilibrium state, thereby sealing the opening of the microwell with a semi-permeable barrier. A lysis reagent and/or other reagents can then be added, with passing of the semi-permeable barrier, thereby lysing the captured single cell and releasing mRNA, which binds to the oligonucleotide molecules of the functionalized particle. Then, buffer exchange can be implemented to shrink the functionalized particle, allowing the functionalized particle to be released from the partition, with captured mRNA material and/or derivative materials, for downstream analysis and processing. Alternatively, buffer exchange can swell the functionalized particle to a sufficient degree that the particles are forced out of the partition (e.g., with captured biomolecules and/or derivatives thereof) making the particles available for collection or other downstream processing.

In another example, the opening of a microwell can have a ridge (e.g., a protruding ring radially into an open surface of the well). After shrinking and loading of the hydrogel particle into the well, the particle can be swelled and made more buoyant. The particle will then make contact with the ridge and the particle will create a seal with the microwell at the ridge. The ridge can contact particle attachment motifs to prevent escape of the swelling hydrogel.

Other applications can be implemented using such functionalized particles that can operate as semi-permeable membranes.

### 5. CONCLUSION

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

1-20. (canceled)

21. A composition for biological sample processing, comprising:
a morphology-adjustable particle comprising a crosslinked polymer that changes size in response to a change of an environmental parameter ; and
a plurality of functional molecules coupled to the particle, each functional molecule comprising a barcode region.

22. The composition of claim 1, wherein the crosslinked polymer comprises a hydrogel.

23. The composition of claim 1, wherein the particle is capable of transitioning from a first state having a first size to a second state having a second size greater than the first size, optionally wherein the particle is further capable of transitioning to a third state having a size smaller than the first size.

24. The composition of claim 1, wherein the environmental parameter is selected from the group consisting of hydration conditions, dehydration conditions, osmolarity, electric field, pH, temperature, pressure, chemical conditions, enzymatic conditions, irradiation conditions, magnetic field, salt conditions, polymer content, and osmotic pressure.

25. The composition of claim 1, wherein the particle is positioned within a microwell of a substrate.

26. The composition of claim 5, wherein the particle, in a swollen state, has a diameter greater than a diameter of an open surface of the microwell.

27. The composition of claim 5, wherein the particle, in a swollen state, protrudes from an open surface of the microwell to contact a tissue sample.

28. The composition of claim 5, wherein the particle, in a swollen state, expands into a flow path adjacent to the microwell to restrict fluid flow.

29. The composition of claim 1, wherein the particle is dissolvable in the presence of a reducing agent that breaks crosslinks within the polymer.

30. The composition of claim 1, wherein the molecules are oligonucleotides for capturing nucleic acids from a cell.

31. A method for processing biological samples, comprising:
providing a plurality of morphology-adjustable particles, each coupled to an oligonucleotide molecule having a barcode region, wherein the morphology-adjustable particles comprise a crosslinked polymer;
co-capturing a morphology-adjustable particle with a cell containing a target molecule within a partition of a plurality of partitions;
binding the target molecule to the oligonucleotide molecule; and
inducing a morphological change of the particle by changing an environmental parameter.

32. The method of claim 11, wherein the environmental parameter is selected from the group consisting of hydration conditions, dehydration conditions, osmolarity, electric field, pH, temperature, pressure, chemical conditions, enzymatic conditions, irradiation conditions, magnetic field, salt conditions, polymer content, and osmotic pressure.

33. The method of claim 11, wherein the partitions are microwells of a substrate.

34. The method of claim 11, wherein the target molecule is an mRNA molecule, and the method further comprises reverse transcribing the mRNA molecule to form a barcoded cDNA molecule bound to the particle.

35. The method of claim 14, wherein inducing the morphological change of the particle releases the particle with the barcoded cDNA molecule from the partition.

36. The method of claim 11, wherein the particles are provided in a first morphological state having a first size, and wherein the inducing step transitions the particle to a second morphological state having a second size greater than the first size.

37. The method of claim 11, wherein inducing the morphological change causes transitioning the particle from a folded state to an unfolded state, or comprises dissolving the particle.

38. The method of claim 11, wherein the partition is positioned adjacent to a channel, and wherein the step of inducing the morphological change of the particle restricts flow within the channel.

39. The method of claim 11, wherein the cell is part of a tissue sample, and wherein inducing the morphological change of the particle causes the particle to protrude from the partition to contact the tissue sample, the method further comprising sequencing the bound target molecule to determine the spatial position of the bound target molecule in the tissue sample.

40. The method of claim 11, further comprising: (a) releasing the particle from the partition; (b) dissolving the particle by exposing the particle to a reducing agent that breaks crosslinks within the polymer; or both (a) and (b).
